(19) United States
(12) Patent Application Publication
(10) Pub. No.: US 2023/0233821 A1
(43) Pub. Date: Jul. 27, 2023

(54) INGESTIBLE DEVICE HAVING A SPIKE ASSEMBLY

(71) Applicant: Novo Nordisk A/S, Bagsvaerd (DK)

(72) Inventors: Morten Revsgaard Frederiksen, Koebenhavn K (DK); Brian Jensen, Broenshoej (DK)

(21) Appl. No.: 18/009,173

(22) PCT Filed: Jun. 10, 2021
(86) PCT No.: PCT/EP2021/065729

§ 371 Date: Dec. 8, 2022

Related U.S. Application Data
(60) Provisional application No. 63/038,148, filed on Jun. 12, 2020.

(30) Foreign Application Priority Data
Jun. 24, 2020 (EP) 20182005.7

(51) Int. Cl. A61M 31/00; A61K 9/48

(52) U.S. Cl. CPC A61M 31/002; A61K 9/4808

(57) ABSTRACT

Capsule devices, such as devices suitable for swallowing into a lumen of a body lumen, are generally provided. In some embodiments, the capsule device (100) comprises a tissue interfacing component (130) disposed relative to a capsule housing (110,120). The capsule device (100) may comprise a plurality of elongated spike members (182, 130) advanceable relative to the capsule housing (110,120) from a non-advanced first state to an advanced second state, wherein free ends of the elongated spike members (182, 130) engages tissue of a lumen wall of the body lumen. In some embodiments, the capsule device (100) comprises an actuator (155) coupled to the plurality of elongate spike members (182) and an energy source (140) coupled to the actuator (155) for moving the plurality of elongated spike members (182, 130) to the advanced second state. At least one of the plurality of spike members (182, 130) is configured as a deflectable spike member (182) that is deflected laterally as the deflectable spike member is advanced toward the second state so that the plurality of spike members (182, 130) act by spreading or pinching engagement on tissue between the plurality of spike members, thereby anchoring the capsule device (100) relative to the lumen wall.

The present invention relates to medical devices, including systems for drug delivery, adapted for introducing into a lumen of a patient and capable of providing anchoring of the medical device relative to a lumen wall.

### BACKGROUND OF THE INVENTION

In the disclosure of the present invention reference is mostly made to the treatment of diabetes by delivery of insulin, however, this is only an exemplary use of the present invention.

Many people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise, since protein-based drugs will be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Prior art references relating to oral dosing of active agents and addressing one or more of the above challenges include WO 2018/213600 A1 and WO 2017/156347 A1. WO 2019/222572 discloses a capsule with first and second compartments wherein the first compartment comprises a mechanism configured to release a component contained within the second compartment. Example components to be contained in the second compartment include an unfolding device, and a self-righting system.

Ingestible capsules have been proposed for both delivery of a therapeutic agent and/or for obtaining medical relevant data from an ingested capsule. For certain applications, it is of primary importance to have the device retained at a target location during a minimum time span to ensure effective utilization of the device.

Having regard to the above, it is an object of the present invention to provide a medical device for introduction into a lumen of a patient, which to a high degree effectively and reliably retains the device relative to a lumen wall, and which obtains the retaining action both in a cost-effective manner and in a minimally invasive and safe manner.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a capsule device suitable for swallowing into a lumen of a gastrointestinal tract of a patient is provided, the lumen having a lumen wall. The capsule device comprises:
a capsule housing sized to be inserted into the lumen,
a tissue interfacing component disposed relative to the capsule housing, the tissue interfacing component configured to interact with the lumen wall at a target location, and
a plurality of elongated spike members arranged advanceable relative to the capsule housing, each spike member extending from a base end to a tissue engaging end opposite the base end, wherein the spike members are configured movable from a non-advanced first state arranged generally within the capsule housing and into an advanced second state wherein the tissue engaging ends of the spike members extend from the capsule housing to engage into tissue at respective locations adjacent to the target location,
an actuator coupled with the plurality of spike members and having a first configuration and a second configuration, wherein the plurality of spike members are retained in the non-advanced first state when the actuator is in the first configuration, and wherein the plurality of spike members are configured to be advanced from the non-advanced first state to the advanced second state by the actuator shifting from the first configuration to the second configuration, and
an energy source coupled to the actuator, wherein the energy source drives the actuator upon actuation,
   and
   wherein at least one of the plurality of spike members is configured as a deflectable spike member comprising a deflectable end portion disposed at the tissue engaging end, wherein the deflectable end portion is deflected laterally as the deflectable spike member is advanced toward the second state so that the plurality of spike members act by spreading or pinching engagement on tissue between the plurality of spike members thereby anchoring the capsule device relative to the lumen wall.

In some embodiments, the capsule device is configured as a self-righting capsule having a geometric center and a center of mass offset from the geometric center along a first axis, wherein when the capsule device is supported by the tissue of the lumen wall while being oriented so that the centre of mass is offset laterally from the geometric center the capsule device experiences an externally applied torque due to gravity acting to orient the capsule device with the first axis oriented along the direction of gravity to enable the tissue interfacing component to interact with the lumen wall at the target location. The capsule device, i.e. with the capsule housing, the tissue interfacing component and the actuator, at least while the actuator assumes the first configuration, performs as a self-righting capsule capable of automatically orienting the capsule device with the first axis oriented generally along the direction of gravity.

In other alternative embodiments, the capsule device is configured as a self-orienting capsule but wherein the capsule device is configured not to rely on gravity induced self-righting when the capsule self-orients relative to a supporting tissue wall.

In some embodiments, either wherein the capsule device defines a self-righting capsule or wherein the capsule device defines a self-orienting capsule, the actuator and the energy source are arranged within the capsule, e.g. within an interior hollow space defined by the capsule housing.

In some embodiments the lumen is the stomach of a subject user, wherein the capsule device is configured for deployment in the stomach lumen, so that the spike members anchor the capsule device relative to tissue wall of the stomach.

In other embodiments the lumen is either a lumen of the small or large intestine of a subject user, wherein the device is configured for deployment in the intestinal lumen, so that the spike members anchor the capsule device relative to tissue wall of either small or the large intestine. In some embodiments, the actuator is movable along a firing axis from the first configuration to the second configuration. In particular embodiments, said firing axis may be coaxial with the first axis.

In different embodiments, the number of deflectable spike members are provided as one, two, three, four or more deflectable spike members.

In exemplary embodiments, the deflectable spike members have a length so that the deflectable spike members are formed to extend 1-3 mm from the capsule housing when the spike members assume their advanced position. Alternatively, the spike members are formed so that the spike members extend a distance selected between 3-5 mm or 5-7 mm from the capsule housing.

In exemplary embodiments, at least one of the one or more deflectable spike members is/are arranged non-detachably relative to the capsule housing, so that at least while the plurality of spike members act by spreading or pinching engagement on tissue said at least one of the one or more deflectable spike members is/are non-separable from the capsule housing.

Exemplary thicknesses of the deflectable spike members, in the direction of deflection, may be selected between 0.1-1.0 mm, such as between 0.2-0.5 mm.

In some embodiments, the tissue interfacing component is coupled with the actuator, such that the tissue interfacing component is retained in a non-advanced first state when the actuator is in the first configuration, and such that the tissue interfacing component is advanced from the non-advanced first state to an advanced second state by the actuator shifting from the first configuration to the second configuration. In other embodiments, the tissue interfacing component is fixedly mounted relative to the capsule housing.

In certain embodiments, the tissue interfacing component is formed as an elongated spike or rod-shaped member having a tissue penetrating end being formed to enable the tissue interfacing component to penetrate tissue upon application of force from the actuator.

The tissue interfacing component of the capsule device may in some embodiments be arranged so that, when the actuator assumes the first configuration and/or the second configuration, at least a part of the tissue interfacing component intersects with the first axis. In embodiments where the tissue interfacing component is formed as an elongated spike or rod-shaped member, when the actuator assumes the first configuration and/or the second configuration, the tissue interfacing component may be arranged so that the tissue interfacing component is oriented coaxially with the first axis. In some further embodiments the tissue interfacing component is fixedly arranged relative to the capsule housing and may include a generally flat surface area configured for interfacing with the tissue of the lumen wall while the plurality of spike members act by spreading or pinching engagement on tissue between the plurality of spike members.

In embodiments where the tissue interfacing component is formed as an elongated spike or needle the tissue interfacing component of the capsule device may define one of the plurality of elongated spike members. Also, the capsule device comprises said at least one deflectable spike member that engages tissue together with the tissue interfacing component to thereby act by spreading or pinching engagement on tissue between the at least one deflectable spike member and the tissue interfacing component.

In further embodiments at least one or each deflectable spike member is configured to engage a spike deflection geometry associated with the capsule housing so that the deflectable end portion of the deflectable spike member is directed laterally as the deflectable spike member moves from the non-advanced first state to the advanced second state. The deflection geometry may be formed to slidingly guide and deflect the respective spike member at least for part of the movement of the spike member from the non-advanced first state to the advanced second state.

In some embodiments, the actuator is arranged for deployment along the first axis towards the tissue interfacing component. In other embodiments, the actuator is movable along an axis which is not parallel with the first axis, but angled with respect thereto.

In some forms of the capsule device, for the plurality of spike members assuming the non-advanced first state, the plurality of spike members extend in parallel or substantially parallel with the first axis.

In further forms, the actuator couples to the base end of the plurality of spike members, either directly or indirectly via one or more additional components. In still further forms, the actuator is formed unitarily with one or more of the plurality of elongated spike members.

At least one deflectable spike member, or all deflectable spike members, may be formed to comprise a portion of a deflectable foil material or may be made fully by a deflectable foil material.

In some variants two or more deflectable spike members are made by foil material common to the two or more deflectable spike members.

In some variants, at least one deflectable spike member, and optionally all of the deflectable spike members, is/are configured so that the tissue engaging end of the deflectable spike member is moved laterally less than 3 mm, such as less than 2 mm, such as less than 1 mm as the deflectable spike member is advanced from the non-advanced first state to the advanced second state.

Said foil material may be provided or comprise a material that is at least partially dissolved when subjected to a biological fluid, thereby disabling anchoring of the capsule device relative to the lumen wall after one of a predefined time interval subsequent to swallowing and a pre-defined time interval subsequent to actuation of the actuator.

Alternatively, or in combination herewith, the spike deflection geometry may comprise a material that dissolves when subjected to a biological fluid to eliminate said spreading or pinching engagement on tissue, thereby disabling anchoring of the capsule device relative to the lumen wall after one of a predefined time interval subsequent to swallowing of the capsule and a predefined time interval subsequent to actuation.

In various forms of the capsule device the tissue interfacing component comprises at least one of a diagnostic unit and a therapeutic unit. The diagnostic unit may be configured as a monitoring device for obtaining medical data from the environment surrounding the capsule device.

In some embodiments, the tissue interfacing component comprises a therapeutic payload configured to provide release of at least a part of the therapeutic payload to the lumen wall at the target location.

The therapeutic payload may in some embodiments be configured for being introduced from the capsule into the lumen wall at the target location by application of force onto the therapeutic payload. In some embodiments, the actuator may be coupled with the therapeutic payload to provide the force to introduce the therapeutic payload from the capsule device into the lumen wall. In some forms the therapeutic payload is directly held by the actuator and moves in synchronism with the actuator.

The tissue interfacing component may in some embodiments comprise a delivery member disposable or disposed in the capsule device, the delivery member being shaped to penetrate tissue of the lumen wall, the delivery member comprising the therapeutic payload or being configured to deliver the therapeutic payload from a reservoir.

In still further embodiments of the capsule device the delivery member is provided as a solid formed entirely from a preparation comprising the therapeutic payload. The delivery member may be rod-shaped and made from a dissolvable material that dissolves when inserted into tissue of the lumen wall to deliver at least a portion of the therapeutic payload into tissue. The rod-shaped delivery member may include a pointed tip end allowing easy penetration into tissue at the target location.

In other embodiments the delivery member is an injection needle having a longitudinal lumen, and wherein the therapeutic payload is provided as a liquid, gel or powder being expellable through the lumen of the injection needle from a reservoir within the capsule.

In still further embodiments, the therapeutic payload comprises an array of micro-needles, such as micro-needles that dissolve when subjected to a biological fluid.

In some forms of the capsule device comprising a plurality of spike members, the plurality of spike members are associated with, such as forming part of, a sealing compartment, wherein the sealing compartment sealingly accommodates the delivery member when the actuator assumes the first configuration, and wherein the delivery member protrudes exteriorly from the sealing compartment when the actuator assumes the second configuration. For such forms, the delivery member may be provided as a member that dissolves when subjected to a biological fluid.

In certain embodiments, the capsule device comprises a firing mechanism configured for actuating the actuator to enable the energy source to drive the actuator upon occurrence of a pre-defined condition. In some embodiments, the pre-defined condition is provided as one or more of a pre-defined condition in the lumen, a set of pre-defined conditions in the lumen, or a signal received by the capsule device and emitted by an external controlling device. In still further embodiments, the firing mechanism comprises an environmentally sensitive mechanism. The firing mechanism may be configured so that in a pre-firing configuration the firing mechanism retains the actuator in the first configuration, and in a firing configuration releases the actuator for enabling the energy source to drive the actuator from the first configuration to the second configuration.

In some forms of the firing mechanism, the capsule housing and the actuator comprises at least one pair of a latch and a retainer portion structured to maintain the actuator in the pre-firing configuration. For each pair of latch and retainer portion the capsule device defines a dissolvable firing member, the dissolvable firing member being at least partially dissolved in a fluid, such as a biological fluid, a retainer portion comprised by one of the capsule housing and the actuator, and a deflectable latch comprised by the other of the capsule housing and the actuator. The deflectable latch is configured for lateral movement relative to the firing axis, and the deflectable latch defines a first surface with a blocking portion, and a support surface disposed oppositely to the first surface and configured for interacting with the dissolvable firing member. In the pre-firing configuration, the blocking portion of the deflectable latch engages the retainer portion in a latching engagement, and the support surface of the deflectable latch interacts with the dissolvable firing member to restrict movement of the deflectable latch thereby preventing release of the latching engagement. In the firing configuration wherein the dissolvable firing member has become at least partially dissolved, the deflectable latch is allowed to move thereby releasing the latching engagement between the blocking portion of the deflectable latch and the retainer portion to allow the energy source to drive the actuator.

By this arrangement, instead of having a dissolvable member that carries the whole power or load of the energy source, the dissolvable part is designed to simply block a mechanical activation system. The mechanical activation system may be designed to rely on parts made from a suitable high-strength material, such as plastic, and do not leave any undissolved pieces that potentially could jam the mechanical activation system.

In exemplary embodiments, the deflectable latch is configured for radial movement relative to the firing axis. In some examples the firing axis and the actuator movement is linear. In other exemplary embodiments, the firing axis may be not linear, e.g. the firing trajectory of the actuator may be arcuate or curved, or may include arcuate or curved trajectories. In accordance herewith, the latch may be configured for lateral movement relative to the trajectory of the actuator to release the actuator.

In exemplary embodiments a plurality of pairs of latch and retainer portions, such as two, three, four, five or more pairs of latch and retainer portions are provided, the pairs of latch and retainer portions being disposed equally around the axis.

In some embodiments said dissolvable firing member is common to all pairs of latch and retainer portions.

In further embodiments, the dissolvable firing member is arranged along the axis, wherein the at least one pair of latch and retainer portion is disposed radially outside of the dissolvable firing member.

In other variants one or more dissolvable firing members is/are disposed, such as in a ring-shaped configuration around the axis, wherein the one or more dissolvable firing members encircle the at least one pair of latch and retainer portion.

The capsule may comprise one or more openings to allow a biologic fluid, such as gastric fluid, to enter the capsule housing for dissolving the dissolvable firing member(s).

In some embodiments, the energy source is or comprises at least one spring configured as a drive spring. Exemplary spring configurations include one or more compression springs, torsion springs, leaf springs or constant-force springs. The spring(s) may either be strained or configured for being strained for powering the actuator. Other non-limiting exemplary types of energy sources for the actuator include compressed gas actuators or gas generators. In some embodiments, in the pre-firing configuration, the energy source exerts a load onto the actuator thereby biasing the actuator along the firing axis. In other embodiments the energy source is configured to exert a load onto the actuator only upon triggering of a trigger member or mechanism of the capsule device.

Whereas this disclosure mainly refers to drug delivery from a capsule device to a lumen or lumen wall, the invention in its broadest aspect is not limited to drug or substance delivery, but rather concerns a particular concept for anchoring an ingestible capsule device relative to a wall of a lumen with the gastrointestinal tract. The capsule device comprising the spike assembly according to the present invention may be configured for other uses. Non-limiting uses include obtaining one or more samples from a body lumen, e.g. by including a sample taking device for introducing a sample from a body lumen or lumen wall into the capsule device, and delivering a monitoring or analysis device, e.g. by disposing or positioning a sensor device from the capsule device into the lumen or lumen wall.

As used herein, the terms “drug” or “payload” is meant to encompass any drug formulation capable of being delivered into or onto the specified target site. The drug may be a single drug compound or a premixed or co-formulated multiple drug compound. Representative drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more of these and other drugs.

### BRIEF DESCRIPTION OF THE DRAWINGS

In the following embodiments of the invention will be described with reference to the drawings, wherein:

FIG. 1 shows a cross-sectional front view of a first embodiment of a capsule device 100 in accordance with the invention with the device assuming a pre-firing configuration, the references mainly referring to the actuation mechanism,

FIG. 2 shows the same cross-sectional front view as FIG. 1, but with references mainly referring to spike assembly 180,

FIGS. 3a through 3d each shows a cross-sectional front view of the first embodiment of the capsule device 100, each view corresponding to a respective state of the device during operation,

FIGS. 4a and 4b are bottom and top perspective views of spike assembly 180 of the first embodiment,

FIGS. 4c through 4e are bottom, side and top plan views of spike assembly 180 of the first embodiment,

FIG. 5a shows schematically a cross-sectional perspective view representing a second embodiment 100′ and a third embodiment 200′ capsule device, the view showing the device in a pre-firing configuration,

FIG. 5b shows schematically a cross-sectional perspective view representing the second embodiment 100′ and the third embodiment 200′ capsule device, the view showing the device in a fired configuration,

FIG. 6a shows schematically a cross-sectional perspective view representing a fourth embodiment 100″ and a fifth embodiment 200″ capsule device, the view showing the device in a pre-firing configuration, and

FIG. 6b shows schematically a cross-sectional perspective view representing the fourth embodiment 100″ and fifth embodiment 200″ capsule device, the view showing the device in a fired configuration.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

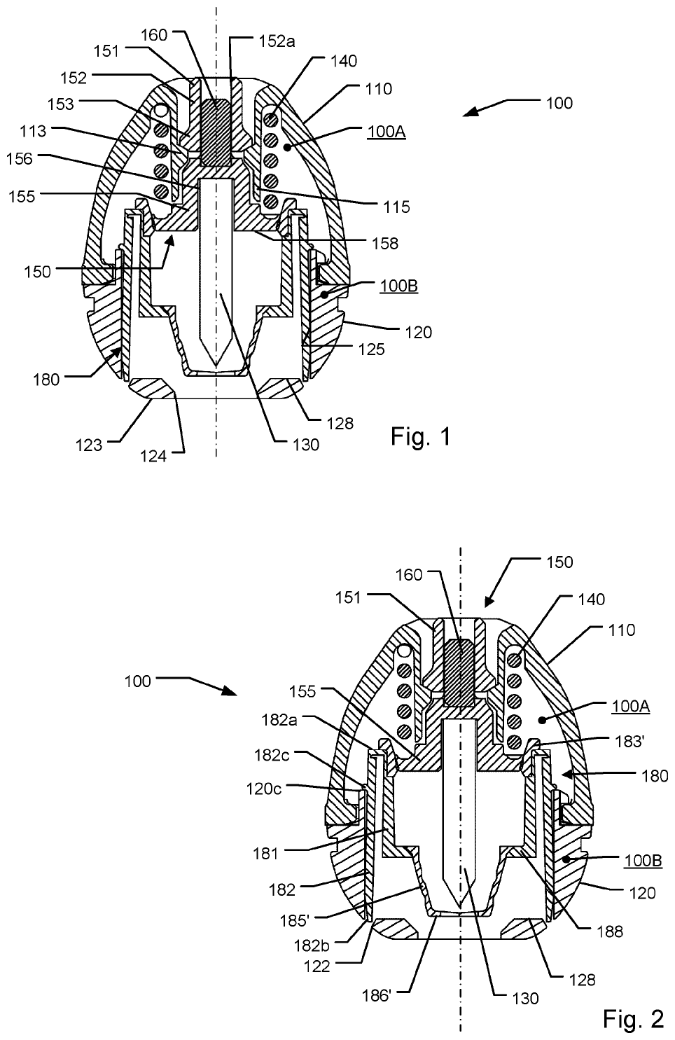

With reference to FIGS. 1 and 2 a first embodiment of a drug delivery device in accordance with an aspect of the invention will be described, the embodiment being designed to provide a capsule device 100 being configured for being ingested into a lumen having a lumen wall, for anchoring relative to tissue at a target location so as to enable a tissue interfacing component to interface with the tissue situated adjacent to the capsule device. In the first embodiment the tissue interfacing component is provided as a solid dose, wherein by deployment of the solid dose from the capsule device allows for substance delivery into the lumen wall. The disclosed embodiment relates to a capsule device 100 suitable for being ingested by a patient to allow the capsule device to enter the stomach lumen, subsequently to orient relative to the stomach wall, and finally to deploy a solid dose payload for insertion at a target location in tissue of the stomach wall. For the capsule device 100 the general principle for orienting the capsule relative to the stomach wall may utilize any of the principles disclosed in WO 2018/213600 A1. FIGS. 1 and 2 both show the first embodiment capsule device 100 in the same view with the device assuming the same state. FIG. 1 mainly refers to parts associated with the actuation mechanism, whereas FIG. 2 mainly refers to a so-called spike assembly to be described further below.

The ingestible self-righting capsule device 100 comprises a first portion 100A having an average density, a second portion 100B having an average density different from the average density of the first portion 100A. The capsule device 100 accommodates a therapeutic payload 130 for carrying an agent for release internally of a subject user that ingests the article. In the shown embodiment, the average density of capsule device prior to deployment is larger than that of gastrointestinal fluid, enabling the capsule device to sink to the bottom of the stomach lumen. The outer shape of the self-righting article is a gomboc shape, i.e. a gomboc-type shape that, when placed on a surface in any orientation other than a single stable orientation of the shape, then the shape will tend to reorient to its single stable orientation. Deployment of the payload is provided by actuation of an actuation mechanism.

The capsule device shown includes an upper (proximal) capsule part 110 which mates and attaches to a lower (distal) capsule part 120. The upper capsule part 110 and the lower capsule part 120 together forms a shell/capsule housing of the device. The capsule defines an interior hollow which accommodates the payload portion provided as an API needle 130, in the shown embodiment formed as a solid dose thin rod-shaped member having a pointed end configured for being forced into tissue. The capsule device 100 further incorporates a spike assembly 180 performing both as an anchoring mechanism and an API sealing chamber for the API needle 130 to maintain the API needle fluidically isolated from the exterior prior to actuation. Upon actuation of the capsule device, the spike assembly 180 provides for anchoring the capsule device relative to tissue at a target location and for allowing the API needle 130 to protrude outside the API sealing chamber. An actuator 150 coupled to an energy source (drive spring 140) is furthermore arranged within the capsule device 100 for generating movement of the API needle 130 and deployment of the spike assembly 180 relative to the capsule parts 110/120.

The API needle 130 is oriented along a firing axis and configured for movement along the firing axis. In the shown embodiment, the upper and lower capsule parts 110, 120 form rotation symmetric parts being symmetric around the firing axis. In the drawings, the device is oriented with the firing axis pointing vertically, and with the API needle 130 pointing vertically downwards towards an exit hole 124 arranged centrally in the lower capsule part 120, the exit hole allowing the pointed end of API needle 130 to be transported through the exit hole and moved towards the exterior of the capsule device 100. The lower part 120 includes a tissue engaging surface 123 which is formed as a substantially flat lower outer surface surrounding the exit hole 124.

Regarding suitable materials for the capsule parts for the embodiment shown in FIGS. 1 and 2, the upper part may suitably be made from a low-density material, such as polycaprolactone (PCL), whereas the lower part 120 may be suitably made from a high-density material, such as 316L stainless steel.

In the shown embodiment, due to the density distribution of the entire capsule device 100, and due to the exterior shape of the device, no matter how the capsule device 100 is oriented initially, the capsule device will tend to orient itself with the firing axis substantially perpendicular to the surface (e.g., a surface substantially orthogonal to the force of gravity, a surface of a tissue such as the wall of the gastrointestinal tract). Hence, the capsule device tends to orient relative to the direction of gravity so that the tissue engaging surface 123 faces vertically downward.

The interior of the lower capsule part 120 includes a sleeve shaped axial guiding structure 125 which extends concentrically with the firing axis from around midway in the capsule interior towards an actuator stop surface 128 defined by an inner bottom surface formed in the lower capsule part 120, i.e. a proximally facing stop surface. Further, in the shown embodiment, a second sleeve shaped structure 115 extends concentrically with the firing axis from the proximal end of the upper capsule part 110 and downwards along the firing axis. The second sleeve shaped structure 115 serves as a retainer structure for retaining the actuator 150 against the drive force emanating from an energized drive spring 140, i.e. the energy source, arranged within the capsule. In the shown embodiment, the retainer structure has a radially inwards protruding retainer portion 113 arranged at a location along the second sleeve shaped structure 115. In the shown embodiment, the retainer portion 113 is provided as two opposed radially inwards protruding arc-shaped protrusions.

In the first embodiment shown in FIGS. 1 and 2, API needle 130 defines a solid delivery member formed partly or entirely from a preparation comprising the therapeutic payload. In the shown embodiment, the solid delivery member is formed as a thin cylindrical rod shaped to penetrate tissue of the lumen wall, the cylindrical rod having a tissue penetrating end and trailing end opposite the tissue penetrating end. The tissue penetrating end of the rod is pointed to facilitate easy insertion into tissue of the lumen wall whereas the trailing end, in the shown embodiment, defines a truncated cylinder cut off by a 90-degree cut. A non-limiting example of a drug suitable for delivery by capsule device 100 is dried compressed API such as insulin.

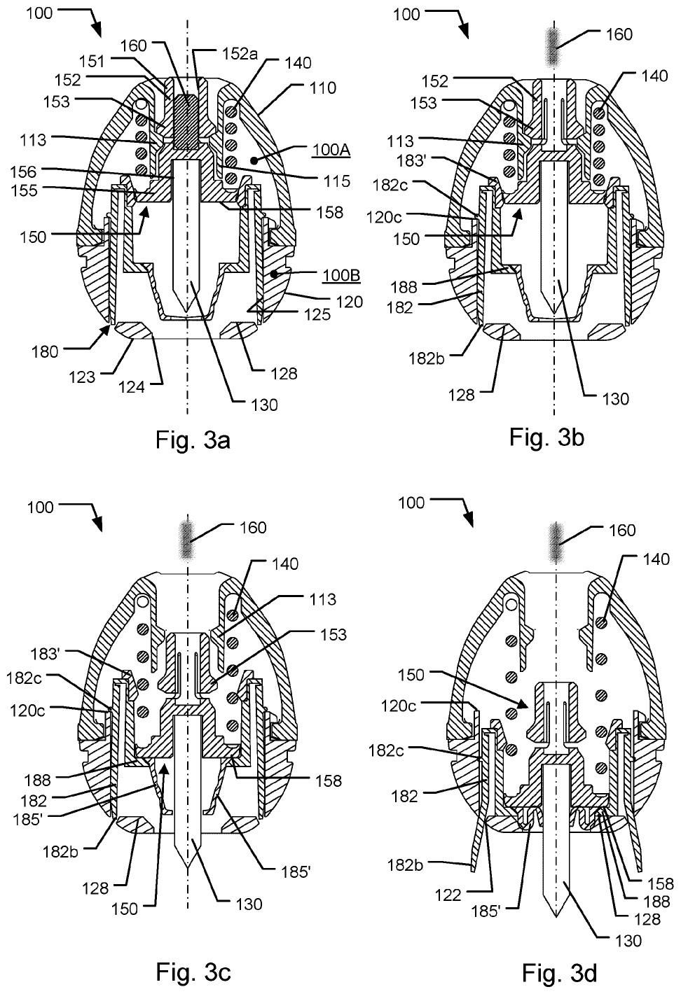

The actuator 150 comprises an upper retaining part 151 and a lower interface part 155 configured for holding the trailing end of the API needle 130 in place. In the shown embodiment, the interface part includes a downward open bore that receives the trailing end of the API needle 130 in a way so that the API needle 130 is firmly attached within the bore. The lower interface part 155 further defines an annular outer flange having a diameter somewhat smaller than the diameter of the axial guiding structure 125 to allow the spike assembly to be located between the outer flange of the lower interface part 155 and the axial guiding structure. In the shown embodiment, the actuator 150 is movable, while being guided for axial movement by the axial guiding structure 125, from a pre-firing configuration shown in FIGS. 1 and 2 to a fired configuration shown in FIG. 3d.

With regard to the above-mentioned drive spring 140, in capsule device 100, a helical compression spring is arranged coaxially with the firing axis. The proximal end of drive spring 140 is seated against a spring seat of upper capsule part 110, i.e. located radially between the second sleeve shaped structure 115 and the outer shell of upper capsule part 110. The distal end of drive spring 140 is seated against a spring seat formed by a proximal surface of the flange defined by the lower interface part 155 of the actuator 150. As part of assembling the capsule device 100 the drive spring 140 has been energized by axially compressing the drive spring 140 between the two spring seats. Hence, the actuator is maintained initially under load from drive spring, such as in the order of 10-30 N. Alternatives to using a compression spring for generating the drive force, other spring and drive configurations may be used to energize the capsule device 100, such as incorporating a torsion spring, a leaf spring, a constant-force spring or similar. In further alternatives, a gas spring or a gas generator may be used.

The upper retaining part 151 of the actuator 150 includes deflectable latches provided in the form of two deflectable arms 152 which extend in distal direction from the upper end of the actuator towards the exit opening 124, each arm being resiliently deflectable in the radial inwards direction. The end of each deflectable arm 152 includes a blocking portion 153 protruding radially outwards from the resilient arm. In the pre-firing configuration shown in FIGS. 1 and 2, a distal surface of each of the blocking portions 153 engage a proximal surface of a corresponding one the retainer portions 113. As the blocking portions 153 initially are located proximally to the retainer portions 113 the actuator 150 cannot be moved distally past the retainer portions 113 unless the deflectable arms 152 become sufficiently deflected in the radially inwards direction.

Referring mainly to FIG. 1, in the pre-firing configuration, a dissolvable pellet 160 is arranged between the two deflectable arms 152 so that radial opposing surfaces of the pellet 160 engage a radially inwards facing support surface of the two deflectable arms 152. In the shown embodiment, the pellet 160 is arranged in a compartment inside the upper capsule part 110, and a proximally arranged upper opening in upper capsule part 110 facilitates fluid exposure to the dissolvable pellet when the capsule device is submerged in a fluid. In the pre-firing configuration shown in FIGS. 1 and 2, as the dissolvable pellet 160 assumes a non-compressible state, the pellet prevents the two deflectable arms from bending inwards. However, upon exposure to a fluid, such as gastric fluid present in the stomach of a patient, the dissolvable pellet starts to dissolve. The pellet 160 is designed to become gradually dissolved so that after a predefined activation time, the pellet has been dissolved to a degree allowing the two deflectable arms 152 to become sufficiently deflected inwards enabling the blocking portions 153 of actuator 150 to be moved distally past the retainer portions 113. In this condition, i.e. the firing configuration, the actuator 150 becomes fired with the load of the drive spring 140 forcing the actuator 150 distally towards the exit hole 124. The actuator 150 drives the API needle 130 distally with the payload tip protruding initially from the capsule, and gradually pressing out the entire or main portion of the API needle 130. The forward movement of the API needle 130 is halted when actuator 150 bottoms out in the lower capsule part 120. This condition is depicted in FIG. 3d and will be described later.

In the shown embodiment, the interface between the retainer portions 113 and the blocking portions 153 is sloped/inclined by approximately 30° so that the deflectable arms will slide inwards when the dissolvable pellet is dissolved. The angle determines the shear forces on the pellet and to which degree the deflectable arms will tend to slide inwards when subjected to the load force. In connection with the acceleration length of the actuator when fired, the optimal angle is 0°, but it requires a much higher spring force to activate such configuration. For the sloped portions, in other embodiments, angles other than 30° may be used.

In the shown example of actuator 150 the upper retaining part 151 is formed as a chamber having radially inwards facing surfaces 152a and wherein the dissolvable pellet 160 is received within the chamber having a tight fit. In the shown embodiment, the central upper part of capsule device 100 includes a single opening for introducing stomach fluid within the capsule. In other embodiments, the capsule may include other design of fluid inlet openings such as multiple openings distributed around the capsule. In some designs, such as in the first embodiment, the API needle 130 is accommodated in a chamber that is initially fluidly sealed from the chamber of the dissolvable pellet and also initially sealed relative to fluid which may enter into capsule device 100 through exit hole 124. In other embodiments no such sealing chamber is incorporated in the designs.

For the dissolvable member discussed above, i.e. the dissolvable pellet 160 forming a dissolvable firing member, different forms and compositions may be used. Non-limiting examples include pellets made from Sorbitol or Microcrystalline cellulose (MCC). Other non-limiting examples include injection moulded Isomalt pellets, compressed granulate Isomalt pellets, compressed pellets made from a granulate composition of Citrate/NaHCO3, or compressed pellets made from a granulate composition of Isomalt/Citrate/NaHCO3. A non-limiting exemplary size of a dissolvable pellet is a pellet which at the time of manufacturing measures Ø1×3 mm.

In situation of intended use, the API needle 130 is inserted into tissue of the lumen wall where it will anchor generally in a direction along the firing axis. FIGS. 3a through 3d show cross sectional side views of the first embodiment of capsule device 100, each view representing the device in different states during actuation. Operation of the device will be described in depth further below.

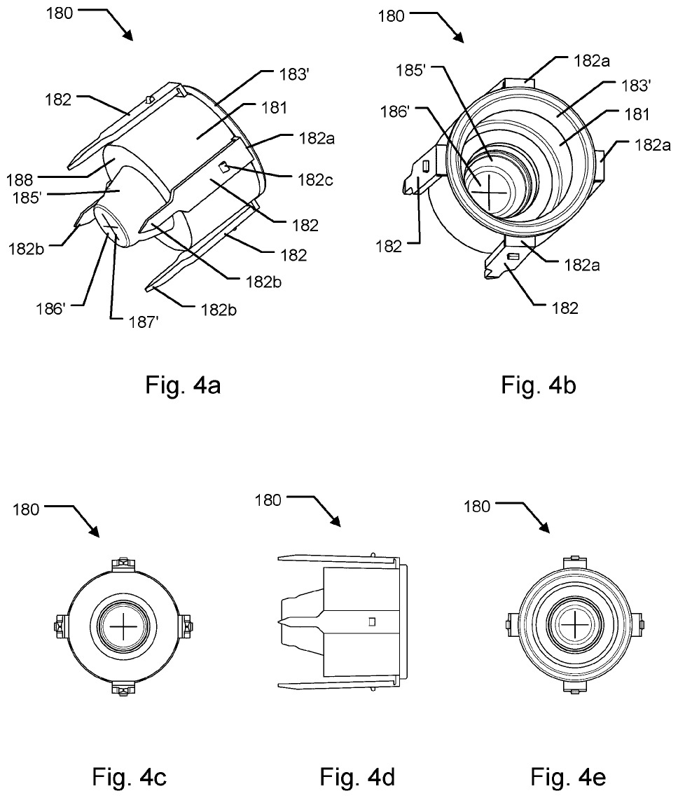

Turning now to FIGS. 4a-4e, the above-mentioned spike assembly 180 is shown in various perspective and plan views. As noted above, the spike assembly 180 performs both as an anchoring mechanism and an API sealing chamber for the API needle 130, the latter being obtained in combination with the lower interface part 155 of the actuator 150.

In this first embodiment the spike assembly 180 is formed as a co-molded two-part component having a first group of material portions made of a thin but relatively stiff material and second group of material portions made from a soft elastic material, such as a rubbery or elastomeric material. In the shown embodiment, the first group of material portions is made from a material which is biodegradable. The first group of material portions generally forms a cylindrical body 181 which makes up the main part of the API sealing chamber and spike members formed integrally therewith. In this embodiment four elongated spike members 182 connects to the proximal upper rim part of the cylindrical body 181 on the radially outwards side thereof. The four spike members 182 are distributed equally around the circumference of cylindrical body 181 so that two spike members pair-wise oppose each other. Each spike member extends from a base end 182a connected to the cylindrical body 181 and generally oriented in the distal direction towards a pointed end 182b located sufficiently far from the base end 182a to enable the pointed end 182b (the tissue engaging end) to be advanceable relative to the lower capsule part 120 through openings formed therein. Located on the radially outwards side of each spike member a detent protrusion 182c protrudes radially outwards to cooperate with an edge section 120c of lower capsule part 120 (cf. FIG. 2).

A soft material portion, formed by the second group of material portions, is arranged as a circumferential lip seal 183′ at the proximal upper rim part of the cylindrical body 181 configured to cooperate with the flange part of lower interface part 155 of the actuator 150 to sealingly engage when the capsule device 100 assumes the pre-firing configuration. The sealing engagement is however disengaged upon triggering of the actuator of the capsule device.

A further soft material portion, also formed by the second material group of material portions, is disposed at the distal lower rim part of the cylindrical body 181 and is formed as an enclosure 185′ for accommodating the distal portion of the API needle 130 with the pointed end of the API needle pointing towards a distal end wall 186′ of enclosure 185′. The distal end wall 186′ has a central portion arranged intersecting the firing axis. In the shown embodiment, the end wall 186′ has a pre-slit but fluid tight opening/shutter 187′ formed therein, the opening/shutter 187′ being formed as a cross 187′. The pre-slit opening/shutter is formed to be fluidically sealed prior to actuation but allows the API needle 130 to easily protrude through the opening upon firing of the capsule device 100. As indicated in FIG. 3d, in the embodiment shown, the enclosure 185′ is formed as a bellows which is able to axially collapse upon actuation of the device so as to fold in an almost flat meandering shape.

Each spike member 182 is designed with sufficient rigidity to allow shallow penetration or piercing engagement with tissue at the target location as the spike members are moved from a non-advanced position to an advanced position relative to the lower capsule part 120. However, the dimensions of spike members are provided in the form of a film material which may assume a generally straight configuration but may be deflected into a curved configuration along the extension of the spike member upon cooperation with a guiding structure. Generally, the spike members are deflectable laterally, meaning that mainly their free ends are able to be moved laterally, such as in the radial direction. In the shown example, the spike members 182 each assumes a generally linear shape that extends generally parallel with the firing axis when not being deflected by other structure. In other embodiments, the spike members may be formed to slightly extend radially outwards towards their pointed ends, such as curved away from the firing axis, when the spike members are not deflected by other structure.

Returning to the operation of the capsule device 100, reference is now made to FIG. 3a which shows the initial state which represents the state the capsule device assumes during storage or just after ingestion. In this state, the actuator 150 assumes the pre-firing configuration where the two deflectable arms 152 are maintained in the shown position by engagement with the undissolved pellet 160 engaging the radially inwards facing support surface 152a of the two deflectable arms 152. As the blocking portions 153 of the deflectable arms 152 are initially located proximally to the retainer portions 113 the actuator 150 cannot be moved distally past the retainer portions 113 even though the drive spring 140 exerts its full load onto the actuator 150.

In this state the spike assembly 180 assumes an initial proximal position relative to the capsule parts 110/120 so that the spike members 182 are located in their non-advanced position with no or only a minor fraction of the spike members extending outside the lower capsule part 120. The actuator 150 engages the circumferential lip seal 183′ to keep this interface fluid tight. Also, the enclosure 185′ with the opening/shutter 187′ closed maintains the API needle enclosure fluid tight. The detent protrusions 182c extending radially outwards from the spike members 182 engage a corresponding proximal edge section 120c of lower capsule part 120 to releasably maintain the entire spike assembly 180 in its initial proximal position.

After ingestion of capsule device 100, the capsule device quickly sinks to the bottom of the stomach. Upon being supported by the stomach wall, due to the self-righting ability of the capsule device, the capsule device will quickly reorient to have its tissue interfacing surface 123 engaging the tissue stomach wall with the firing axis of the capsule device oriented virtually vertical, i.e. with the API needle pointing downwards. In FIG. 3b, which schematically indicates that dissolvement of pellet 160 has begun due to exposure to gastric fluid entering the opening or openings in upper capsule part 110, the support from pellet 160 against the deflectable arms 152 is discontinued at a specific time after swallowing. The load of the drive spring 140 will cause the deflectable arms 152 to be deflected radially inwards to allow the blocking portions 153 to pass the retainer portions 113 enabling the actuator to be fired in the distal direction.

As shown in FIG. 3c, the reluctance of the detent protrusions 182c disengaging the cooperating proximal edge section 120c is greater than the reluctance of actuator 150 disengaging the circumferential lip seal 183′. Hence, the actuator will start distal movement relative to the spike assembly 180 which is still arrested relative to the capsule lower part 120. During this movement lower interface part 155 of actuator 150 is slidingly guided along the radially inwards facing surface of cylindrical body 181 as the API needle 130 protrudes through opening/shutter 187′ and gradually inserts the tip portion of API needle 130 into tissue of the lumen wall. In FIG. 3c, a distal surface 158 bottoms out relative to a flange part 188 of the spike assembly 180 and the full force from drive spring 140 acts upon the spike assembly 180 biasing the spike assembly distally. Due to the disengagement between the flange of the lower interface part 155 of actuator 150 and the circumferential lip seal 183′ the most proximal portion of cylindrical body 181 and the spike members 182 are not supported at their radial inwards facing surfaces. The distal biasing force on the spike assembly 180 and the elastic properties of the spike assembly 180 cause the spike members 182 and the cylindrical body 181 becoming deflected inwards to cease the arresting engagement between detent protrusions 182c and proximal edge section 120c. Hence, the drive spring 140 subsequently drives the spike assembly 180 in the distal direction slaved by the distal movement of the actuator 150.

FIG. 3d shows a further state wherein the spike assembly 180 and the actuator 150 has moved further distally until a state wherein a distal facing surface of flange part 188 of the spike assembly 180 has reached a proximal facing surface 128 of lower capsule part 120 thus preventing further movement of spike assembly 180 and actuator 150. As the actuator has moved distally from the state shown in FIG. 3c to the state shown in FIG. 3d, the spike assembly 180 has been slaved for synchronized distal movement with the actuator 150, the synchronized distal movement occurring relative to the capsule parts 110/120. This has caused the spike members 182 to move distally relative to the openings formed in lower capsule part 120 to cause the spike members 182 to move from the non-advanced position to the advanced position. In the embodiment shown, the lower capsule part 120 includes a guiding surface formed by edges 122 associated with each spike member, each edge 122 cooperating by engaging with the radially inwards facing surface of the spike member 182. Due to the radial location of the edges 122 relative to the radial location of the base end 182a of the spike members 182, and relative to edges of the radially inwards facing surface 125, the tissue engaging end 182b of each spike member is urged radially outwards as the spike assembly 180 moves distally from the position shown in FIG. 3c to the position shown in FIG. 3d. This has the effect that each tissue engaging end 182b of the four spike members 182 will gently engage the tissue by initially shallowly pierce or poke into the soft tissue at their respective location. As the spike assembly 180 moves further distally, the relative distance between opposingly arranged spike members 182 will gradually increase serving to spread the tissue located between opposed pairs of spike members. This has the result of providing a gripping effect on tissue located between the pairs of opposed spike members 182 and in this way serving to temporarily attach the capsule device 100 relative to the tissue of the stomach wall for a time sufficient for the API needle to deliver a major portion of the API due to absorption into tissue.

At some point in time the fired capsule device will release from tissue. In the shown example, the first group of materials of the spike assembly 180 is made from a biodegradable material which gradually degrades upon exposure to a biologic fluid, and the spike members 182 will gradually loose rigidity tending to release the gripping effect on tissue with time. Exemplary, non-limiting embodiments, depending on the specific application of a particular capsule device, suitable times for release of the capsule device relative to the tissue may be selected such as 10 minutes, 20 minutes, 30 minutes, 1 hour or even longer. In addition, for example due to movements, such as due to food impacting the capsule device 100, the capsule device may be released from tissue prior to the spikes becoming degraded. Subsequently, the remaining parts of the capsule device will travel out through the digestive system of the user and be excreted. In the shown embodiment, the spike members 182 are formed to extend 1-3 mm from the capsule part 120 when the spike members assume their advanced position. Depending on the particular application for a capsule device according to the invention, for non-limiting embodiments, other lengths of spike members 182 may be chosen so that the spike members extend a distance selected between 3-5 mm or 5-7 mm from the capsule part 120. The thickness in radial direction of the spike members may be chosen between 0.1-1.0 mm, such as between 0.2-0.5 mm.

in the shown embodiment, the material forming the spike members may include portions which gradually becomes softer or dissolve after a specific time after being exposed to gastric fluid to thereby cease the anchoring effect. In other embodiments, where the spike members are not degradable, the release of the capsule device from the tissue may rely on the dissolution of the API needle becoming dissolved to a certain degree to cause the capsule device to release attachment relative to tissue. In still other embodiments, the guide surfaces of the capsule housing that cooperate with the spike members, such as the edges 122 of the first embodiment, may include material portions which are configured for gradually becoming dissolved after being exposed to gastric fluid to cause the spreading effect of the spike members to cease for release of the capsule device subsequent to a particular exposure time.

In the first embodiment the spike members 182 are molded in a way which makes the spike members extend substantially parallel to the firing axis when the spike members are not deflected by other structures. In other embodiments of capsule devices, the spike members are molded to exhibit a non-biased form where each spike member extends radially away from the cylindrical body 181 when the spike members are not deflected by other structures, i.e. in a spread configuration. When assembling such capsule device the spike members are forced to enter into a non-spread configuration inside the capsule device. In such embodiments, upon actuation of the capsule device, the tissue engaging end 182b of the spike members will tend to automatically spread relative to each other, due their inherent bias towards returning to the spread configuration, as the spike members are advanced out of the capsule, optionally without any guiding being required for obtaining the spreading effect. It should also be noted that, in still other embodiments, the spike members are designed so as to pinch tissue between pairs of spike members so as to provide a retaining effect for the capsule device relative to tissue at the target location.

In the shown first embodiment, the number of deflectable spike members are four that are equally distributed. In other embodiments, the number of spike members may be provided as 1, 2, 3, 5, 6 or even more deflectable spike members distributed in a particular pattern. It is to be noted for an embodiment which includes only a single deflectable spike member, in embodiments which additionally comprises a needle shaped member that is forced into tissue, such as the shown API needle 130, the needle shaped member may perform as an additional spike member which cooperates with the deflectable spike member to obtain the spreading or pinching effect on tissue located between the spike members, thus also obtaining the effect of tissue anchoring.

As described above for the first embodiment, the capsule device 100 incorporates a spike assembly 180 performing both as an anchoring mechanism and an API sealing chamber for the API needle 130 to maintain the API needle fluidically isolated from the exterior prior to actuation. It is to be noted however, for different aspect of the invention, in accordance with different further embodiments of capsule devices, an assembly for only obtaining a single one of the two functions may be provided, so that an assembly corresponding to spike assembly 180 either omits the API sealing chamber function or the anchoring/spiking function.

In different embodiments of the capsule device according to the invention, the attachment between the API needle and the actuator may be obtained by using a friction or press fit. Alternatively, an adhesive may be used at the interface, such as sucrose. Still alternatively, the attachment may be obtained by initially wetting the API needle and utilizing inherent stiction between the actuator and the API needle. In certain embodiments, in situation of use, upon the actuator reaching its final destination, detachment between the API needle 130 and the actuator 150 may occur, such as at the interface between the API needle and the actuator upon exposure to gastric fluid for a specified duration, allowing the needle to be dislodged in the tissue wall enabling further dissolution of the API needle in the tissue, i.e. independent and separated from the remaining capsule device. In other embodiments, a desired detachment may be obtained by detaching a major portion of the API needle from the remaining API needle being still adhered or fastened to the actuator. In some embodiments, the API needle includes a weakened point which determines the point of separation. In still further embodiments, as discussed further below, the actuator and the API needle may be formed as a unitary component all made of a composition containing API, and wherein the intended API needle to be pushed out from capsule device is separated from the actuator portion. Also, in alternative embodiments, the payload may act as an actuator by itself to cooperate directly with the release mechanism and the energy source.

In the first embodiment described above, the spike assembly 180 is maintained fixedly retained axially relative to the capsule parts 110/120 as the actuator 150 moves a first distance distally until the spike assembly 180 is released from the bottom capsule part 120. In other embodiments, the capsule device may be configured to move the spike assembly and the actuator in synchronism, at least for an initial part of the stroke that the actuator experiences. In certain embodiments, the spike assembly is initially slaved with the actuator for a first part of the actuator stroke, whereas for a second part of the actuator stroke, the spike assembly 180 becomes arrested relative to the capsule parts 110/120 while the actuator continues to push the API needle further into tissue, i.e. moves further distally subsequent to the spreading of pinching effect of the spike members have been fully established.

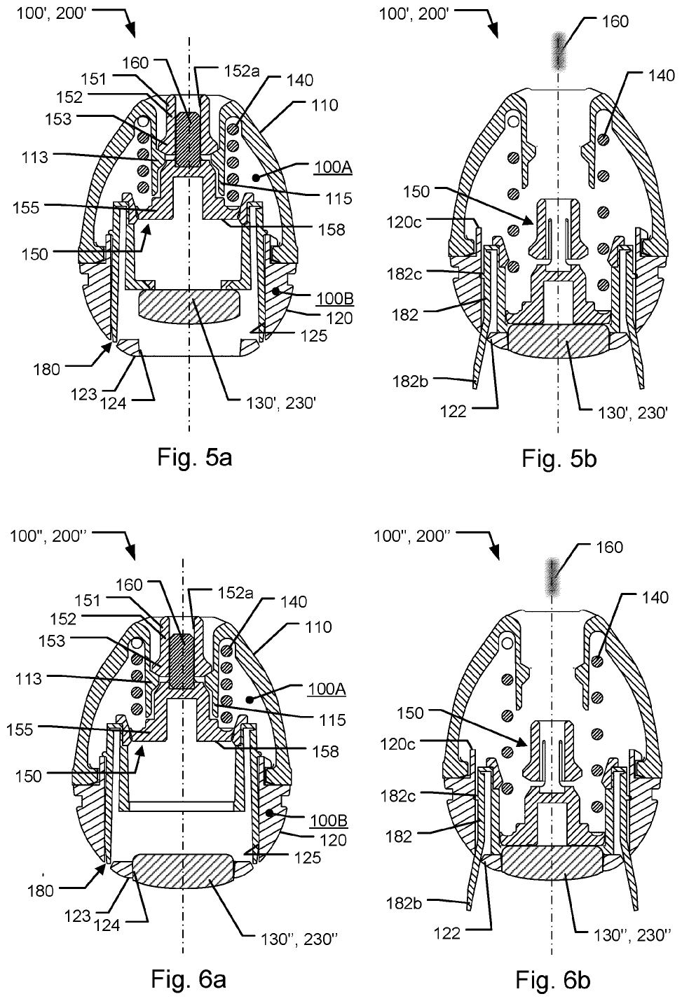

Now turning to FIGS. 5a and 5b, the views shown schematically are representative for both a second embodiment 100′ and a third embodiment 200′ of exemplary capsule devices. The second and third embodiments generally correspond to the capsule device 100 of the first embodiment, but wherein the tissue interfacing component, i.e. the API needle has been replaced by either a tablet-shaped API portion 130′ or a monitoring device 230′ for obtaining medical data from the environment surrounding the capsule device. The view shown in FIG. 5a generally corresponds to the view shown in FIG. 3a, i.e. the state that the capsule device 100′/200′ assumes in the pre-firing configuration, i.e. during storage or prior to ingestion. The view shown in FIG. 5b generally corresponds to the view shown in FIG. 3d, i.e. the state that the capsule device 100′/200′ assumes in the fired configuration, i.e. after the movement of the actuator has ended.

In the second embodiment 100′, the tablet shaped API portion 130′ is arranged to follow movement of the spike assembly from the position shown in FIG. 5a to the position shown in FIG. 5b. In FIG. 5b the tablet gets in intimate contact with the tissue situated below the capsule device 100′. The spike assembly 180 serves for retaining or anchoring the capsule device 100′ relative to the tissue to ensure intimate contact with the tissue for the API portion 130′ to have a major portion of the API becoming absorbed in tissue. In further embodiments, the API portion 130′ may be provided to incorporate an array of micro-needles configured for mucosal delivery of the API of the API portion 130′.

In the third embodiment 200′, monitoring device 230′ is arranged to follow movement of the spike assembly from the position shown in FIG. 5a to the position shown in FIG. 5b. In FIG. 5b the tablet becomes located in contact or adjacent the tissue situated below the capsule device 200′. The spike assembly 180 serves for retaining or anchoring the capsule device 200′ relative to the tissue to ensure retainment at least for an interval suited to obtain medical data by means of monitoring device 230′.

FIGS. 6a and 6b, further show schematic views that are representative for both a fourth embodiment 100″ and a fifth embodiment 200″ of exemplary capsule devices. The fourth and fifth embodiments generally correspond to the capsule devices according to the second and third embodiments shown in FIGS. 5a and 5b, but wherein either a tablet-shaped or micro-needle equipped API portion 130″, or a monitoring device 230″ for obtaining medical data from the environment surrounding the capsule device is incorporated at a fixed location relative to capsule parts 110/120. Hence, in such embodiments, the actuator 150 only serves to move the spike assembly 180 for obtaining the desired retainment or anchoring effect of the capsule device relative to the tissue at the target location.

Although the above description of exemplary embodiments mainly concern ingestible capsules for delivery in the stomach, the present actuation principle generally finds utility in capsule devices for lumen insertion in general, wherein a capsule device is positioned into a body lumen for attachment relative to the wall of the body lumen. Non-limiting examples of capsule devices may include capsule devices for intestinal delivery of a drug either by delivery into the intestinal lumen or into the tissue wall of an intestinal lumen. For drug delivery, delivery may be performed using a delivery member, such as a needle, or via micro-needles which is inserted into the tissue wall of a lumen. Alternatively, drug delivery may be performed through one or more exit openings of the capsule device without the use of a delivery member, such as by jet injection into a mucosal lining of a lumen wall.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A capsule device suitable for swallowing into a lumen of a gastrointestinal tract of a patient, the lumen having a lumen wall, wherein the capsule device comprises:
a capsule housing sized to be inserted into the lumen,
a tissue interfacing component disposed relative to the capsule housing, the tissue interfacing component configured to interact with the lumen wall at a target location, and
a plurality of elongated spike members arranged advanceable relative to the capsule housing, each spike member extending from a base end to a tissue engaging end opposite the base end, wherein the spike members are configured movable from a non-advanced first state arranged generally within the capsule housing and into an advanced second state wherein the tissue engaging end of the spike members extend from the capsule housing to engage into tissue at respective locations adjacent to the target location,
an actuator coupled with the plurality of spike members and having a first configuration and a second configuration, wherein the plurality of spike members are retained in the non-advanced first state when the actuator is in the first configuration, and wherein the plurality of spike members are configured to be advanced from the non-advanced first state to the advanced second state by the actuator shifting from the first configuration to the second configuration, and
an energy source coupled to the actuator, wherein the energy source drives the actuator upon actuation,
wherein the capsule device is configured as a self-righting capsule having a geometric center and a center of mass offset from the geometric center along a first axis, wherein when the capsule device is supported by the tissue of the lumen wall while being oriented so that the centre of mass is offset laterally from the geometric center the capsule device experiences an externally applied torque due to gravity acting to orient the capsule device with the first axis oriented along the direction of gravity to enable the tissue interfacing component to interact with the lumen wall at the target location, and
wherein at least one of the plurality of spike members is configured as a deflectable spike member comprising a deflectable end portion disposed at the tissue engaging end, wherein the deflectable end portion is deflected laterally as the deflectable spike member is advanced toward the second state so that the plurality of spike members act by spreading or pinching engagement on tissue between the plurality of spike members thereby anchoring the capsule device relative to the lumen wall.

2. The capsule device as in claim 1, wherein the number of deflectable spike members are one, two, three, four or more deflectable spike members.

3. The capsule device as in claim 1, wherein each deflectable spike member is configured to engage a spike deflection geometry associated with the capsule housing so that the deflectable end portion of the deflectable spike member is directed laterally as the deflectable spike member moves from the first state to the second state.

4. The capsule device as in claim 3, wherein the spike deflection geometry is formed by a material that dissolves when subjected to a biological fluid to eliminate said spreading or pinching engagement on tissue, thereby disabling anchoring of the capsule device relative to the lumen wall after one of a predefined time interval subsequent to swallowing of the capsule and a predefined time interval subsequent to actuation.

5. The capsule device as in claim 1, wherein the actuator is arranged for deployment along the first axis towards the tissue interfacing component.

6. The capsule device as in claim 1, wherein, in the non-advanced first state the plurality of spike members extend substantially parallel with the first axis.

7. The capsule device as in claim 1, wherein the at least one deflectable spike member comprises a portion of a deflectable foil material or is fully made by a deflectable foil material.

8. The capsule device as in claim 1, wherein said spike members include a plurality of deflectable spike members, and wherein the plurality of deflectable spike members are made by foil material common to the plurality of deflectable spike members.

9. The capsule device as in claim 8, wherein the foil material is at least partially dis-solved when subjected to a biological fluid, thereby disabling anchoring of the capsule device relative to the lumen wall after one of a predefined time interval subsequent to swallowing and a predefined time interval subsequent to actuation of the actuator.

10. The capsule device as in claim 1, wherein the tissue interfacing component comprises a therapeutic payload configured to provide release of at least a part of the therapeutic payload to the lumen wall at the target location.

11. The capsule device as in claim 10, wherein the tissue interfacing component comprises a delivery member disposable in the capsule housing, wherein the delivery member is shaped to penetrate tissue of the lumen wall, the delivery member either comprising the therapeutic payload or being configured to deliver the therapeutic payload from a reservoir.

12. The capsule device as in claim 11, wherein the delivery member is coupled to the actuator, wherein the delivery member is retained within the capsule housing when the actuator is in the first configuration, and wherein the delivery member is configured to be advanced from the capsule housing and into the lumen wall by movement of the actuator from the first configuration to the second configuration.

13. The capsule device as in claim 11, wherein the delivery member is a solid formed entirely from a preparation comprising the therapeutic payload, wherein the delivery member is rod-shaped and made from a dissolvable material that dissolves when inserted into tissue of the lumen wall to deliver at least a portion of the therapeutic payload into tissue.

14. The capsule device as in claim 11, wherein the delivery member is an injection needle having a longitudinal lumen, and wherein the therapeutic payload is provided as a liquid, gel or powder being expellable through the lumen of the injection needle from a reservoir within the capsule housing.

15. The capsule device as in claim 11, wherein the deflectable spike member(s) are associated with, such as forming part of, a sealing compartment, wherein the sealing compartment accommodates the delivery member when the actuator assumes the first configuration, and wherein the delivery member protrudes exteriorly from the sealing compartment when the actuator assumes the second configuration.
